(19) United States
(12) Patent Application Publication
(10) Pub. No.: US 2009/0287307 A1
(43) Pub. Date: Nov. 19, 2009

(54) Orthopaedic Implant and Prosthesis Systems, Devices, Instruments and Methods

(76) Inventors: John Parry, Worcestershire (GB); Liang Qian, Redditch (GB)

Correspondence Address: Beusse Wolter Sanks Mora & Maire, 390 N. ORANGE AVENUE, SUITE 2500, ORLANDO, FL, 32801, US

(21) Appl. No.: 12/132,825

(22) Filed: Jun. 4, 2008

(30) Foreign Application Priority Data
May 13, 2008 (GB) GB0808639.9

(51) Int. Cl. A61F 2/08; A61B 17/04

(52) U.S. Cl. 623/13.11; 623/13.2; 623/13.14; 606/301

(57) ABSTRACT

The present invention provides a replacement ligament comprising first and second ends, two or more securing portions for securing said ligament to a portion of bone material and a body portion comprising a first component having a first, higher, elongation per unit load characteristic; and a second component having a second, lower, elongation per unit load characteristic. The first and second components are between said two or more securing portions and arranged in load series such that initial, lower, loading is reacted by said first component and subsequent, higher, loading is reacted by said second component.

### RELATED APPLICATIONS

This application claims priority to British Application No. GB0808639.9 filed 13 May 2008, which is incorporated herein in its entirety.

### FIELD OF THE INVENTION

The present invention relates to orthopaedic implants and/or prostheses, systems of these and instruments and/or methods for their implantation. The invention is applicable to attachment to bone structures, particularly the cervical, thoracic, and lumbar spine but may lend themselves to use in other application such as, in particular, artificial ligaments and the like.

### BRIEF SUMMARY OF THE INVENTION

Bones and related structural body parts, for example spine and/or vertebrae and/or intervertebral discs, may become crushed or damaged as a result of trauma/injury, or damaged by disease (e.g. by tumour, auto-immune disease), or damaged as a result of degeneration through an aging process. Some of the conditions that can arise from such deterioration may be so severe as to require surgical intervention. For instance, collapse of or damage to one or more vertebral bodies or intervertebral discs can result in compression of the spinal cord and/or nerve roots, causing pain, loss of function, and even complete paralysis of the lower limbs, with loss of bladder and bowel control. It is increasingly common for the surgical intervention to involve removal of the affected parts of the structure and replacement with an implant or prosthesis such that stability is restored and the source of pain removed.

### BRIEF DESCRIPTION OF THE INVENTION

Traditionally, the most successful surgical approach in cases of herniated or degenerated intervertebral discs has been for the surgeon to remove the affected disc and insert an implant that provides a means for bone growth through or around a supporting structure, resulting in a permanent bony fusion between adjacent vertebral bodies. More recently, however, spinal implants have been developed that enable a disc to be replaced whilst maintaining motion between the vertebrae. These implants commonly utilise a fixed or mobile bearing to enable relative motion between endplates, which attach to the corresponding surfaces of the vertebrae. Another option is for the implant or prosthesis to consist of an elastomeric core sandwiched between endplates, which permits flexing and twisting movement, with the benefit that the implant or prosthesis does not require separate components with articulating surfaces that may create wear debris.

Whether the surgeon seeks to perform a fusion or motion-preserving intervention, the method of implantation is broadly similar. The spinal column can be accessed from a number of approaches: posterior, lateral, anterior, or antero-lateral. Once the appropriate access is gained, the diseased disc is excised and, if necessary, the vertebral endplates are prepared (osteophytes may need to be removed, or the endplate abraded to encourage osteoblast activity). Then the implant or prosthesis is inserted and secured to the vertebral body, where necessary, with suitable fixation devices. Following a successful implantation, the wound is closed and the patient moved to a recovery area.

In many cases, the anterior approach is the most appropriate, and for some surgeries (such as operations at the Lumbar5/Sacral1 level where the iliac crests prevent alternative approaches) it is the only option. In order for the surgeon to gain access to the affected disc space, the anterior longitudinal ligament is generally severed and/or removed from the area surrounding the intervertebral space. The anterior longitudinal ligament is a tough ligament running the entire length of the spine, attached to the anterior surface of each vertebra and acting as a tension band—preventing hyperextension when an individual extends the spine by bending backwards. Due to the anterior longitudinal ligament's position and relative width, it blocks access to the intervertebral disc space and, as a result, is cut during surgery. Ligaments do not have a blood supply and heal very slowly, so in most instances when the ligament is severed it cannot be repaired.

The absence of an anterior longitudinal ligament can cause complications post-operatively, particularly for motion-preserving implants. Without the resistance to extension that the ligament provides, the patient may hyperextend the spine, generating excessive and localised loading on the posterior aspect of the implant or prosthesis. This can cause damage to the implant or cause the implant to crush into the vertebral body. Alternatively, the loading on the posterior aspect of the implant or prosthesis may force it to move in an anterior direction, potentially pushing part or all of the implant or prosthesis out from the intervertebral space and into the patient's body. Any such movement could result in the implant or prosthesis coming into contact with vital body structures, for instance, the aorta, vena cava, or great iliac vessels, which lie next to the spine. The rupture of any of these vessels could have catastrophic results, including death of the individual.

Various approaches have been taken to provide artificial ligaments to connect bones. For example, U.S. Pat. No. 4,187,558 to Dahlen and Stubstad teaches a surgically implantable skeletal ligament which in an exemplary embodiment comprises a braided multifilament flexible core encased in an elastomeric material, the latter appearing to be protective and not load-bearing. U.S. Pat. No. 4,662,886, to Moorse and Strover, teaches a surgical implant such as for a replacement for a ligament, consisting of a multiplicity of flexible filaments. The arrangement of the filaments is stated to encourage penetration and ingrowth of tissue between the core filaments. U.S. Pat. No. 5,674,296, to Bryan and Kunzler, teaches a multi-component device that is stated to replace a disc between vertebral bodies. In a disclosed embodiment there is included a simple prosthetic longitudinal ligament connected by screws through the device and into adjacent vertebral bodies. Exemplary materials for this strap-like ligament are stated to include a Kevlar-like material or a Goretex-like material. U.S. Pat. No. 6,585,769, to Muhanna and Middleton, teach a flexible prosthesis comprising a flexible elongated plate member that is attached to vertebral bodies by bone screws or other fasteners through slotted apertures in the plate member. It is stated that a single material of the plate member has physical characteristics “approximating the natural biomechanical characteristics of a spinal ligament.” U.S. Pat. No. 6,652,585 to Lange teaches a spinal stabilization system that may be used to connect vertebral bodies. The system comprises a flexible member comprised of mesh like components that include a component corresponding to a direction that corresponds to the direction of the fibers of disc annulus tissue. The embodiments depict the use of mesh layers, singly or overlain, where the mesh includes fibers at right angles. Other patents of interest include U.S. Pat. No. 5,575,819 to Amis, U.S. Pat. No. 5,800,543 to McLeod and Shafghian, and U.S. Pat. No. 5,681,310 to Yuan.

Notwithstanding the various approaches to artificial ligaments, there remains a need in the field for a more effective replacement ligament device and replacement ligament system, one that in particular will advance the art by providing for long term performance from a device that is designed for proper tension and loading on its components.

According to one aspect of the present invention there is provided a replacement ligament comprising:first and second ends, two or more securing portions for securing said ligament to an adjacent body portion; a body portion comprising a first component having a first, higher, elongation per unit load characteristic; and a second component having a second, lower, elongation per unit load characteristic; and wherein said first and second components are between said two or more securing portions and arranged in load series such that initial, lower, loading is reacted by said first component and subsequent, higher, loading is reacted by said second component.

### BRIEF DESCRIPTION OF THE DRAWINGS

Aspects of the present invention will now be described by way of example only with reference to the accompanying drawings, in which:

FIG. 1 provides a typical load-deformation curve of a natural ligament;

FIG. 2 shows an anterior view of a two-hole replacement ligament design;

FIG. 3 shows an anterior view of a two-hole replacement ligament design, with a widened section in-between the two fixation points;

FIG. 4 shows an anterior view of a three-hole replacement ligament design, with a triangular shape;

FIG. 5 shows an anterior view of a three-hole replacement ligament design, with a Y-shape;

FIG. 6 shows an anterior view of a four-hole replacement ligament design, with a rectangular shape;

FIG. 7 shows an anterior view of a four-hole replacement ligament design, with an oval shape;

FIG. 8 shows an anterior view of a four-hole replacement ligament design, consisting of two parallel elements;

FIG. 9 shows an anterior view of a four-hole replacement ligament design, with a “dog bone” shape;

FIG. 10 shows an anterior view of a four-hole replacement ligament design, with an X-shape;

FIG. 11 shows an anterior view of a four-hole replacement ligament design, consisting of two crossed element;

FIG. 12 shows an anterior view of a six-hole replacement ligament design;

FIG. 13 shows an anterior view of a six-hole replacement ligament design intended for use in cases where multiple vertebral discs are being treated;

FIGS. 14 to 17 show perspective views of alternative methods of assembling a grommet into a replacement ligament design;

FIG. 18 shows a perspective view of a polymer fibre ligament inside a matrix of silicone or similar material;

FIG. 19 shows a perspective view of an expanding bollard fixation device assembled to an replacement ligament;

FIG. 20 shows a perspective view of a bone screw fixation device assembled to an replacement ligament;

FIG. 21 shows an anterior view of a four-hole replacement ligament attached to the lumbar spine;

FIG. 22 shows an anterior view of two three-hole replacement ligaments attached to the lumbar spine;

FIG. 23 shows an anterior view of a multiple level replacement ligament attached to three vertebrae of the lumbar spine; and

### DETAILED DESCRIPTION OF EMBODIMENTS OF THE INVENTION

While the teachings of the present invention may be utilized for a wide range of ligament replacements, the discussion in this section focuses on use of the replacement ligament to replace (or to augment) a portion of the anterior longitudinal ligament (ALL). This is because there exists a need for an implant or prosthesis that can be substituted for the portion of the anterior longitudinal ligament that is cut or excised during reconstructive spinal surgery. Functionally, the implant or prosthesis will have to satisfy a primary critical extension-limiting biomechanical performance criteria of the natural anterior longitudinal ligament and a secondary criteria of providing a barrier against implant migration. The primary criteria is achieved by providing in a replacement ligament system of the present invention two related components: a first component providing for a more facile ligament deformation under an initial, lesser load in a first load zone; and a second component providing for extension in a second load zone in which load is greater per unit deformation, wherein the two components cooperatively respond to perform as a replacement ligament. The second criteria is achieved by providing an anti-adherent coating or layer onto one or more surfaces of the implant, as will be discussed in detail later herein.

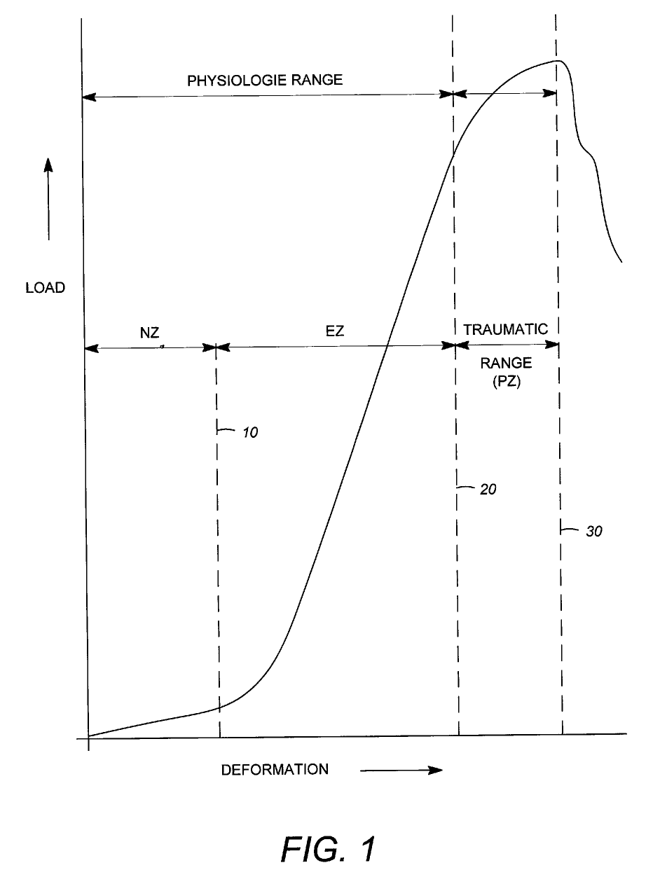

To better appreciate the operation of various embodiments, an example of a typical load-deformation curve for a natural ligament is presented in FIG. 1. As load or stress is increased along the Y-axis, the natural ligament deforms or strains as shown along the X-axis. In a first zone of deformation, identified as the neutral zone (NZ) and extending from the Y-axis to a first borderline 10, there is a relatively high deformation per unit of load as the ligament extends from a relaxed state and any slack is taken up. In a second, adjacent zone, identified as the elastic zone (EZ) and extending between first borderline 10 and a second borderline 20, a relatively higher load is required to deform the ligament per unit of deformation. This may be expressed simplistically in the following equation:

ΔD_{NZ}/ΔL_{NZ}>ΔD_{EZ}/ΔL_{EZ}

where D_{NZ} is the displacement in the neutral zone corresponding to change in L_{NZ}, the load placed on the ligament in the neutral zone, and D_{EZ} is the displacement in the elastic zone corresponding to change in L_{EZ}, the load placed on the ligament in the elastic zone. This second zone represents the normal operating range of the ligament in which a generally linear extension per unit load is obtained and upon unloading the ligament returns to normal length. It is noted that although the first component, in some embodiments, may continue to deform as load increases in the elastic zone, the properties of the second component, and its arrangement in relation to the first component, are such that the second component is responsible for the majority of the load carrying capacity in the elastic zone.

The second borderline 20 marks the end of the normal physiologic range for the ligament. Beyond the normal physiologic range of motion there is a traumatic range, shown as between the second borderline 20 and a third borderline 30, identified as the plastic zone (PZ) where trauma to the ligament may occur, leading ultimately to ligament failure (which may involve plastic deformation, meaning an inability to return to normal shape and performance).

Various embodiments of the present invention comprise independent but related components that may separately provide for the deformations, respectively, of the first NZ and the second EZ. For example, in some embodiments a compressible spacer, positioned in an aperture at an end of the replacement ligament, deforms responsive to the first, lower load range corresponding to the neutral zone of a particular natural ligament which the replacement ligament was designed to replace. In some such embodiments a plurality of woven tensile fibres, in a ligament body of replacement ligament which comprises the aperture, extends responsive to load in the second, elastic zone of the particular natural ligament which the replacement ligament was designed to replace. In other such embodiments the ligament body comprises a non-braided (such as moulded) material that deforms responsive to the second, elastic zone of the particular natural ligament which the replacement ligament was designed to replace.

In other embodiments there need not be a compressible spacer such as described immediately above. In some such embodiments one or more types of filaments in a braided or otherwise woven portion of a ligament body deform(s) responsive to the first, lower load range corresponding to the neutral zone of a particular natural ligament which the replacement ligament was designed to replace. One or more types of other filaments in the ligament body deform(s) responsive to the second, elastic zone of the particular natural ligament which the replacement ligament was designed to replace

Embodiments also may comprise both the first component and the second component in the ligament body in a manner in which the second component does not come under load until the first component has been subjected to most or all of the load in the neutral zone. One approach to this is to provide the second component in the form of tensile fibres that are oriented to have a slack in them in the longitudinal axis between distal ends of the ligament body, such as a wavy pattern, where the deformation in the neutral zone removes most or all of the slack.

In yet other embodiments, as described below, there is at least one region of a replacement ligament body associated with deformation corresponding with the neutral zone, and at least one region of the replacement ligament body corresponding with the elastic zone. Examples of such regions include regions of a braided or woven ligament body, regions of a moulded ligament body, and sections of a multi-piece assembled ligament body.

In various embodiments another aspect of the invention is that one or both of the anterior and posterior surfaces of the replacement ligament (or ligament body in some claims) comprises an anti-adhesive material that imparts anti-adhesive properties. The types of materials and approaches to providing this are provided below. It is appreciated that for some applications an anti-adhesive anterior surface is desired so that adjacent blood vessels do not adhere to the replacement ligament, while it is desired to have structural connective tissue adhere on the posterior surface. In such embodiments an anti-adhesive material is provided on the anterior surface only. However, some surgeons, in some applications, want to grow tissue on the anterior surface of the replacement ligament and not on the posterior side. In such embodiments an anti-adhesive material is provided on the posterior surface only. Finally, in some applications an anti-adhesive material is provided both on the anterior and the posterior surfaces. Alternatively, the coating may be provided as a sheath or wrap of material surrounding the ligament and made from, for example, Goretex™.

Also, in various embodiments the implant or prosthesis has a low profile and can be securely attached to the anterior aspect of patient's spine. Various shapes are provided, many of which additionally function to provide a physical barrier designed to limit movement of the more posteriorly disposed vertebral implant (e.g., a disk replacement prosthesis such as a “cage”) so that this does not slip or displace forward postoperatively. It is recognized that this is more likely in non-fusion arthrodesis procedures. Also, as used herein, the term “replacement ligament” is meant to refer not only to devices of the present invention that completely replace a ligament, such as during a surgical procedure, but also to replacement ligaments that augment, reinforce or supplement an existing ligament, such as when a portion of the original ligament remains after a surgical procedure or accident.

The subject replacement ligament invention incorporates design and functional features relating to the requirements for its successful function. A basic aspect for embodiments of the present invention is for the replacement ligament to provide a stop to limit extension. To this end, the main body of the ligament consists of woven threads or fibres arranged to form a generally rectangular overall shape, and orientated so as to provide longitudinal stiffness under tension. One example of an approach to fabrication of a suitable woven structure may be found in U.S. Pat. No. 5,800,543, which is incorporated by reference for these teachings. In some embodiments, the fibres are made of polyester, but other materials such as polypropylene or poly-ether-ether-ketone (PEEK) fibres could also be used. These fibres act as a tension band when taut, limiting the movement of adjacent vertebral bodies by preventing extension. Due to their woven structure, the fibres allow flexibility under compression and axial rotation, which is advantageous when used in conjunction with a motion-preserving spinal implant.

In an exemplary embodiment, to replace a portion of the anterior longitudinal ligament, in order to provide a means of fixation to the vertebral bodies, the replacement longitudinal ligament comprises a ligament body having a length that includes at least two circular apertures, positioned at opposite ends of the length along the longitudinal centreline. These apertures are sized and positioned according to the fixation method used, which may include bone screws, bollards, or staples. The fixation devices rigidly fix the replacement anterior longitudinal ligament to the anterior surface of each of the two respective vertebral bodies. Each fixation device may include a shaft that is driven or screwed into the vertebral body, and a head (or central cross bar in the case of a staple), the function of which is to hold the replacement anterior longitudinal ligament pressed against the anterior surface of the vertebral body. The head portion is dimensioned such that the replacement anterior longitudinal ligament is prevented from slipping over the anterior end of the shaft during loading or movement of the spinal column. Other components and features of various embodiments are discussed below.

As may be appreciated from the above discussion, an aspect of the replacement anterior longitudinal ligament is that in some embodiments some of the elasticity characteristics of the natural anterior longitudinal ligament may be replicated. Consequently, in such embodiments the replacement anterior longitudinal ligament design incorporates design features that ensure the resistance to extension increases as the anterior aspects of the two adjacent vertebral bodies move apart during extension. Additionally, these or other features ensure that the replacement anterior longitudinal ligament returns to its initial overall length when the tensile loading is removed, limiting the amount of slack present when the spine is in a neutral position or in flexion. One embodiment that addresses this need incorporates compressible inserts, one of which fits into at least one of the circular apertures in the replacement anterior longitudinal ligament body. These inserts, of which washers are one type, and compressible grommets are another type, are compressed between the replacement anterior longitudinal ligament and the shaft of the fixation device under loading, so that the resistance to extension increases in relation to the amount of movement between the adjacent vertebral bodies, and return to their original dimensions as the loading is removed. In another embodiment of the invention, the compressible inserts are pre-installed into the ligament body.

Another embodiment of the design features elastomeric fibres incorporated into the woven structure of the replacement anterior longitudinal ligament body. These elastomeric fibres stretch as the replacement anterior longitudinal ligament experiences loading initially in the neutral zone, until the overall length reaches the maximum dimension, at which point tensile fibres come under load, through the elastic zone, and then come to a tautness after such loading so that the vertebral bodies are restricted from moving further apart due to tensioning of one or both of the elastomeric and tensile fibres. When the tensile loading is removed, the elastomeric fibres return to their original dimensions, restoring the original overall dimensions of the replacement anterior longitudinal ligament. In another embodiment, the entire replacement anterior longitudinal ligament body is woven from elastomeric fibres of suitable material properties.

Due to its intended location in the body in various embodiments, it is vital to ensure that the replacement anterior longitudinal ligament does not cause abrasion of the tissues and vasculature that surrounds it. In such circumstances, the blood vessels can rupture or adhere to the surface of an implanted structure, both of which are highly undesirable outcomes. To address this issue, at least one surface of the subject invention is coated or impregnated with material that will reduce friction and resist adhesion by bodily tissue and vasculature. An example of such a material is polysiloxane, commonly referred to as “silicone”. In one embodiment of the invention, the ligament body of the replacement anterior longitudinal ligament is formed from a silicone sheet. In a further embodiment, the replacement anterior longitudinal ligament is made from a sheet comprising a polysiloxane matrix that is internally reinforced with a plurality of tensile fibres. A surface of the replacement anterior longitudinal ligament that is coated, impregnated, or with a layer of polysiloxane or other material that renders that surface non-abrasive and/or non-adherent, is termed “anti-adherent”.

The use of polysiloxane as an anti-adhesive material is not meant to be limiting. Other examples of materials that may be used to provide anti-adhesion properties to one or more surfaces of the replacement ligament include polyurethane, PTFE, and Goretex™. The application of the anti-adhesive material to provide a desired anti-adhesive surface may be by any convenient method known to those skilled in the art, including but not limited to spraying, brushing, extruding, and incorporation into the weave or into a matrix composition during molding.

The anti-adhesive material may be applied to either one or to both of the anterior-facing and the posterior-facing surfaces of the replacement ligament. Correspondingly, for methods of providing a replacement ligament an anti-adhesive surface may be oriented in a desired orientation, such as only to the anterior side or only to the posterior side of a replacement anterior longitudinal ligament body.

In cases where more than one intervertebral level is affected, the subject invention is designed such that two consecutive levels can be treated without interference between the two respective replacement anterior longitudinal ligaments. This may be achieved by the dimensions of the design locating the fixation points towards the opposite aspects of the shared vertebral body (with the superior replacement anterior longitudinal ligament occupying the superior edge of the shared vertebral body, and the inferior replacement anterior longitudinal ligament occupying the inferior edge of the shared vertebral body), resulting in clearance between the two replacement anterior longitudinal ligaments. Alternatively, the subject invention may be designed with the fixation points lying closer to the midline of the shared vertebral body, but with a shape that enables the superior profile of the inferior replacement anterior longitudinal ligament to tessellate with the inferior profile of the superior replacement anterior longitudinal ligament.

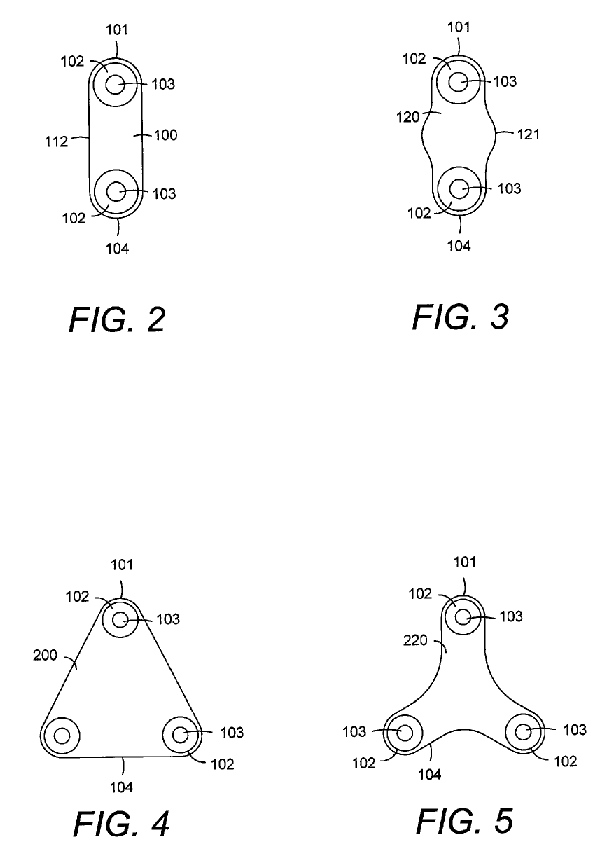

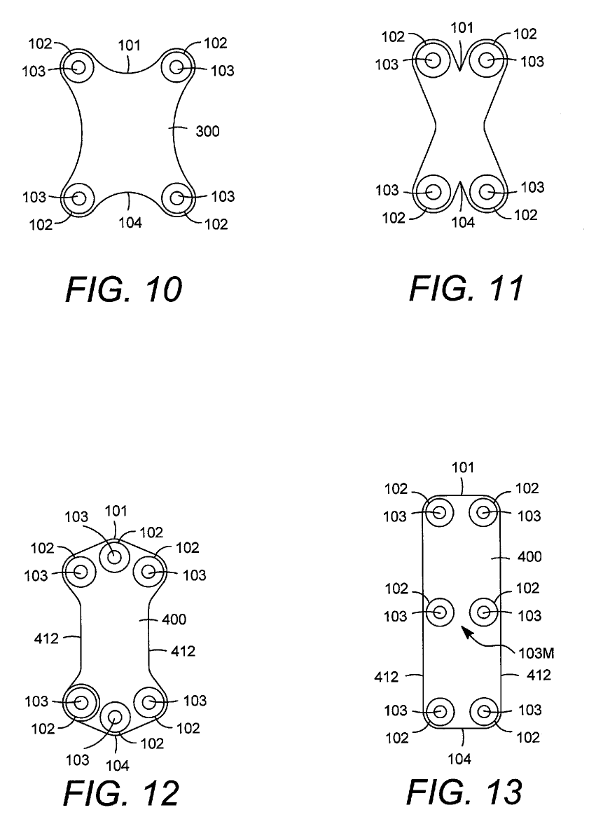

FIG. 2 to FIG. 24 inclusive show figurative illustrations of some possible configurations of the subject invention. It will be understood that no limitation of the scope of the invention is intended by any configuration included or omitted from these diagrams.

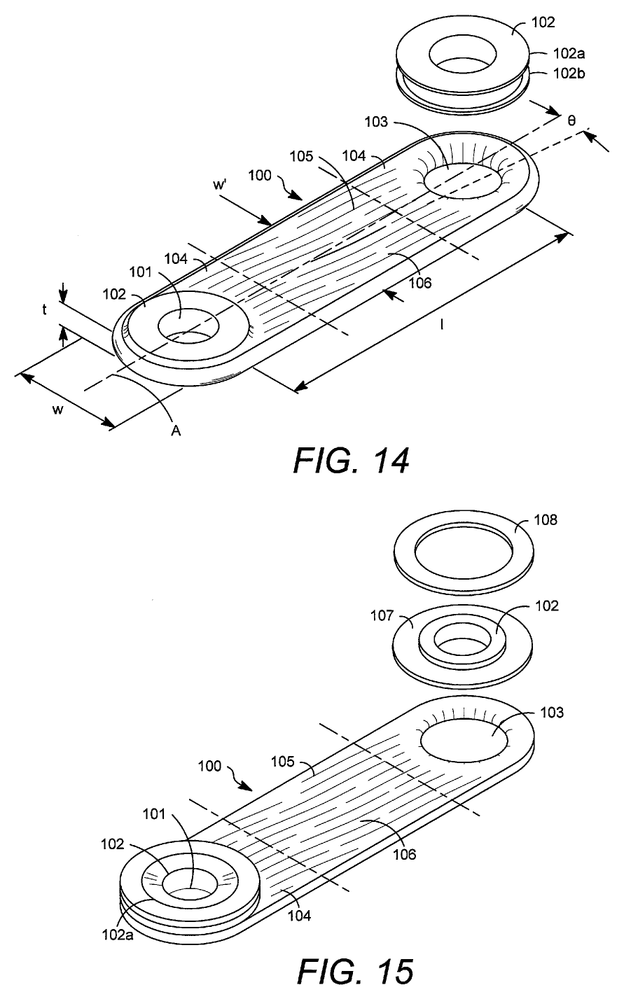

FIG. 2 provides a top surface view of a replacement ligament body 100 having a proximal end 101 and a distal end 104 having parallel sides 112 and apertures 103. The apertures 103 are depicted to have in them grommets 102, but this is not meant to be limiting. This embodiment is discussed in greater detail in the discussion of FIGS. 14 to 18.

FIG. 3 provides a top surface view of a replacement ligament 120 that has a widened central region 121 in the middle of the length between the apertures 103. This widened central region 121 may help retain a disc implant surgically inserted between the bones (not shown) to which the ligament 120 is attached.

FIG. 4 provides a top surface view of a triangular-shaped replacement ligament 200. Among other applications, this may be used effectively when two or more replacement ligaments 200 are placed along three or more adjacent vertebral bones, allowing an overlap of the ligaments 200 and greater distribution of fasteners (now shown, see infra) that are placed in the apertures 201.

FIG. 5 provides a top surface view of a Y-shaped replacement ligament 220. As for the triangular embodiment of FIG. 4, this may be used effectively when two or more replacement ligaments 220 are placed along three or more adjacent vertebral bones, allowing an overlap of the ligaments 220 and greater distribution of fasteners discussed later herein that are placed in the apertures 201.

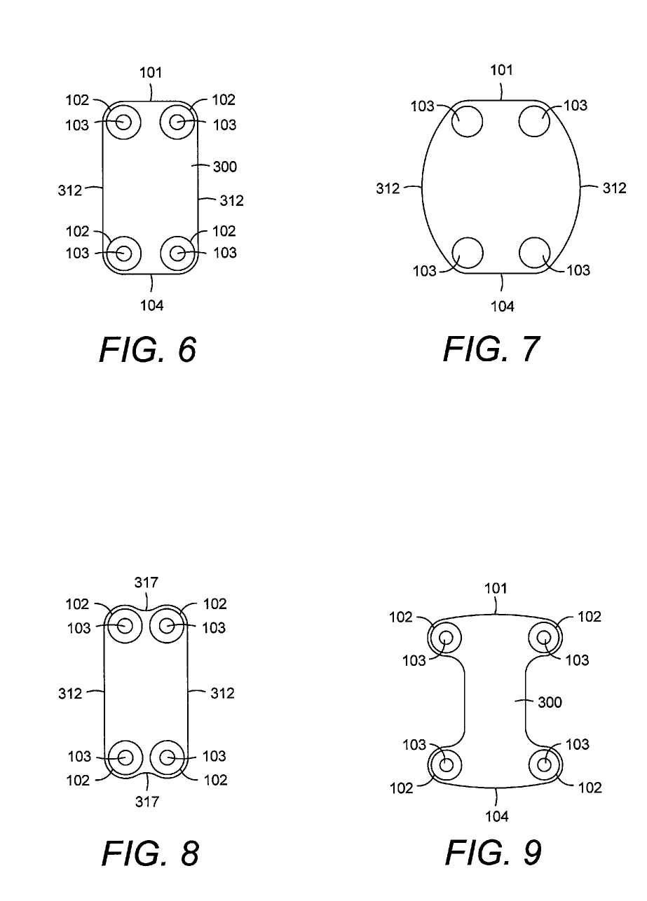

FIG. 6 provides a generally rectangular replacement ligament body 300 comprising parallel lateral sides 312 and two apertures 103 at each end 101, 104. This broader ligament body 300, compared to that of FIG. 2, and the relatively wider attachment span due to the two adjacent apertures 301 at each end, provide for relatively greater resistance to extension from movement by the bones to which the ligament body 300 is attached.

FIG. 7 is similar to FIG. 6 except that the grommets are absent and the lateral sides 312 are not parallel, as in FIG. 3, but rather are convexly expanded outward. This is to provide a greater barrier to retain a disc prosthesis.

FIG. 8 also is similar to FIG. 6, having parallel lateral sides 312 and upper and lower cut-outs 317. Such an arrangement may be made by stitching two ligaments of FIG. 2 together along a common edge.

FIGS. 9 to 11 provide different shapes of ligament bodies 300 that comprise more widely spaced apart apertures 103 at each respective ends 101, 104. These shapes may provide for relatively greater management of rotational movement by the bones to which they are attached.

FIG. 12 depicts a ligament body 400 having parallel lateral sides 412 along a medial portion of the ligament body, and a broadening at the distal ends to accommodate three apertures 103 at each end so as to provide for three fixation devices (not shown) at each end. Such approach may be used to provide for greater strength of attachment, or for an adequate attachment when smaller fixation devices are necessitated.

FIG. 13 depicts a ligament body 400 having parallel lateral sides and three pairs of apertures 401. A medial pair of apertures 103M disposed between the apertures 401 at the respective distal ends 101, 104 may be attached to a bone (not shown) that is medial to bones (not shown) attached via the apertures at the respective distal ends. Thus three bones may be attached with a single ligament body 400. This is not meant to be limiting and more than three bones may be attached with ligament bodies having additional medial apertures.

Referring now to FIG. 14, the ligament body 100 is defined by having a length, l, measured between fixation apertures 103, a width, w, and a thickness, t. The length l is aligned along axis A which may be considered a longitudinal axis between two or more bones of interest, such as the vertebral bodies of the spine. These dimensions are defined by anatomical conditions and the exact values will depend on the chosen shape and intended position within the body. As an illustration, a typical replacement anterior longitudinal ligament used in the lumber spine will be between 30 mm and 60 mm in length, with a width of 15 mm to 25 mm. The natural anterior longitudinal ligament is approximately 3mm thick, and the replacement ligament of the present invention will match this as closely as is practicable. For more complex designs than that shown in FIG. 14, the replacement ligament will also have a midline width, w′, which will be independent from the width at the fixation points. In various embodiments the replacement ligament body 100 comprises a woven mesh of any combination of elastic and/or inelastic fibres, or an elastomeric sheet, which may form a matrix that is internally reinforcement by polymeric fibres. In FIG. 14 are viewable a plurality of tensile fibers 105 of which the ligament body 100 is comprised. In various other embodiments the ligament body 100 may be a monolithic structure or comprised of two or more attachable regions or sections.

Further as to the ligament system as depicted in FIG. 14, the ligament body 100 comprises the tensile fibers or filaments 105 woven so as to cross the longitudinal axis A at an angle θ less than or equal to 30 degrees. In other embodiments this maximum angle between the fibers and the longitudinal axis A is 20 degrees, and in other embodiments this maximum angle is 10 degrees and in preferred arrangements may be zero degrees. Further, as used herein, including the claims, “substantially parallel” is taken to mean being oriented at an angle not exceeding 30 degrees relative to the longitudinal axis A. The angular orientation of the fibres may be employed to advantage when it is desired to provide a different degree of resistance at different angular positions. In this and other embodiments, the ligament body 100 provides a single layer of the woven tensile fibers 105. By “single layer” is meant that the ligament body 100 does not comprise multiple separable or overlain layers, although a woven pattern having a desired thickness and strength, being thicker than a single layer of tensile fibers 105, falls within this definition of a single layer. At each end of the length l of ligament body 100 is an aperture 103, the length so dimensioned as to span two bones which the ligament body is to connect. Each aperture 103 is adapted to receive a fastener, discussed further below, to connect to one of the two bones being connected by the ligament body 100. A compressible grommet 102, which is one example of a compressible insert, is sized to enter the aperture 103 and comprises a fixation aperture 101 sized to receive the fastener (not shown). In this embodiment, the grommet 102 is a first component deformable responsive to a first, lower load range corresponding to a neutral zone of a natural ligament that is being replaced. This is effective to provide a desired load bearing as the bones to which the ligament body 100 is attached move apart and place the ligament body 100 in a first stage of tension that corresponds to the neutral zone of that natural ligament. As may be appreciated, the deformation of the compressible grommet 102 involves compression in a region between the fastener in the fixation aperture and a distal end 109 of the aperture 103, (best seen in FIG. 24 and extension along the lateral sides of the fixation aperture in response to the displacement. In various embodiments, the size and the material for the compressible grommet 102 are selected so that as the load reaches the borderline 10 in FIG. 1, between the neutral zone and the elastic zone, the material under compression in the noted region will not further substantially compress. Under further load the tensile fibers in the ligament body 100, these fibers being a second component, extend responsive to this second, more elevated load in the range corresponding to an elastic zone of the natural ligament. The compressible grommets 102 and the tensile fibers 105 of the ligament body 100 are related in that they are arranged in load series so as to sequentially respond to increasing loads through the NZ and the EZ load ranges of the natural ligament that the ligament system comprising these components replaces. In effect, the grommets 102 are the main load reacting element in the NZ range and the tensile fibres are the main load reacting elements in the NZ range. This provides the ligament with a relatively soft initial extension characteristic followed by a relatively high resistance to elongation in the EZ zone, thereby approximating to the extension profile of a natural ligament.

The fibers of this and other woven embodiments are preferably preconditioned by “preload” methods known in the art to relieve tension and remove undesired slack. This is done to minimize anticipated deformation during use in the body. Generally, an applied preload is applied in a factory such as to exceeds the maximum expected physiological load yet also be below the load that would cause plastic deformation. Relatively higher preloads, still within these limits, may protect against additional fiber settling, which has been reported to possibly occur from infrequent excessive loading while in use in the body.

As may be appreciated, the compressible grommets 102 are fitted into corresponding apertures 103 in the longitudinal end sections 104 of the replacement ligament body 100. An example configuration of grommet is shown in FIG. 14, although it will be appreciated that many more are available and the exact design of compressible grommet 102 will be dependent on the specific requirements of the overall design. It is intended that each longitudinal end section 101, 104 will contain at least one aperture 103. In various embodiments both the grommet apertures 103 and the fixation apertures 101 will be generally circular in profile. The compressible grommets 102 are made from a complementary material to the ligament body 100. For instance, if the replacement ligament body 100 is made from a generally inelastic material, then the grommets 102 will be made from a compressible material, such as silicone which will have a relatively high deformation (compression or extension) per unit load relative to the fibres 105. The grommets themselves 102 may include lips 102a, 102b which protrude beyond the main body thereof and which, in operation, are deformed when inserting said grommet into the aperture 102 and act to retain it therein once inserted.

In some alternative embodiments, such as those described below, if the replacement ligament body 100 comprises both the first and the second components that respond to load in the NZ and the EZ zones, respectively, then the grommets 102 will be made from a rigid material, for instance in some embodiments a biocompatible metal such as titanium alloy or stainless steel, or from an implantable plastic such as UHMWPE or polypropylene. In various embodiments the design and material of the replacement ligament body 100 may mean that the grommets 102 are not required; the fixation apertures 101 being formed directly into the replacement ligament body 100 itself. Alternatively, one may employ one or more compressible grommets 102 at a first end 101 and one or more non-compressible grommets at a second end 104.

In one embodiment, the replacement ligament body 100 is coated or impregnated with anti-adhesive material or materials, if the material or materials forming the general structure of the replacement ligament body 100 do not have sufficient anti-adhesive properties themselves. It is considered by some that anti-adhesive properties are most highly desirable on the anterior surface of the replacement ligament's mid-section 106. In some embodiments of the invention, therefore, the anti-adhesion coating or treatment is applied solely to one surface of the replacement ligament's mid-section 106, or to the entirety of one surface which—when implanted—will be orientated to the anterior direction. In further embodiments, the entire mid-section 106 (both front and back surfaces) is coated or treated to resist adhesion, with the end sections 104 left open. It is contemplated that this embodiment will allow bone growth into the posterior side of the end sections 104, enhancing fixation between the replacement ligament 100 and the vertebral bodies.

FIG. 15 shows an alternative approach for assembling the compressible grommets 102 into the apertures 103 in the replacement ligament body 100. In this embodiment of the subject invention, the compressible grommets 102 each have a radial flange 107, having a diameter greater than the aperture 103, which is positioned above or below the longitudinal end 104 of the replacement ligament body 100 such that a ring 108 may be paced thereover so as to sandwich said compressible ring in position, as best seen at the left hand end of FIG. 15. If the ring 108 s made of suitable material it may be stitched to the ligament by means of a thread as shown in FIG. 17 which is repeatedly passed through said ring, flange 107 and ligament. It is contemplated that this secondary ring 108 may be manufactured from the same material as the replacement ligament body 100, or from an alternative material, as appropriate. In various embodiments once the compressible grommet 102 and the secondary ring 108 are in place, the three components optionally are secured together by a method such as stitching, bonding, or fusing.

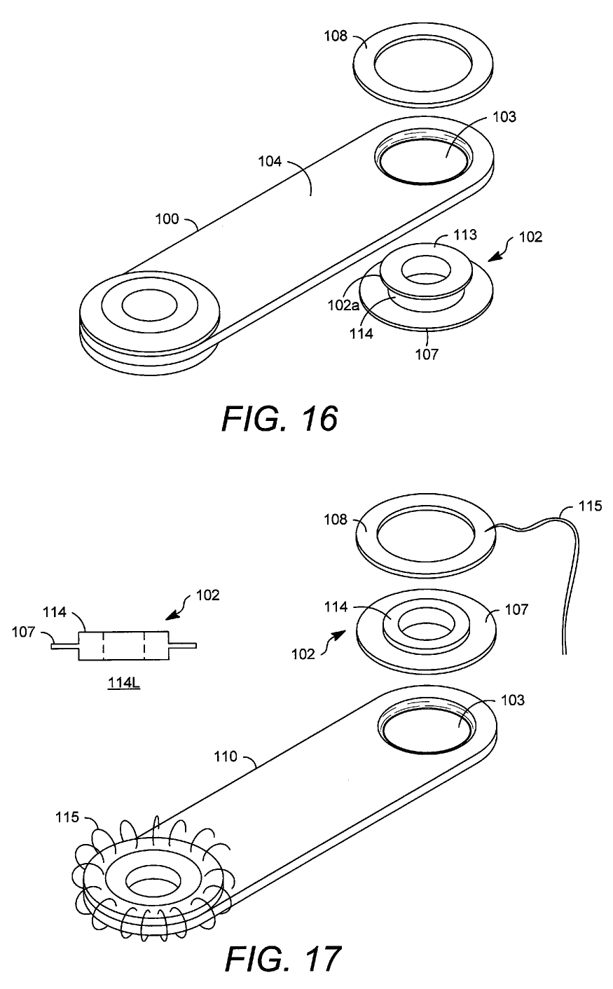

FIG. 16 illustrates a further arrangement of ligament in which the ring 108 is made from compressible material similar or the same as that used for the grommet 102. In such a case, as in others, the grommet may be provided with a lip 102a at an upper or lower surface thereof and being sized such as to allow the ring 108 to pass thereover by deformation whilst providing a degree of resistance to the removal thereof. The lip 102a effectively acts as a “click-fit” arrangement for the ring 108 so as to allow easy assembly whilst also providing good retention. Such retention may allow one to dispense with any stitching or other such systems for maintaining the assembly in position. A fully assembled arrangement is shown at the left hand side of FIG. 16.

FIG. 17 shows the stitching of components together as discussed above with reference to FIG. 15 and also shows a cross-section depiction of a compressible grommet 102 additionally shows an optional lower extension 114L of the central columnar core 114. In such embodiments this optional lower extension 1 14L fits into the aperture 103.

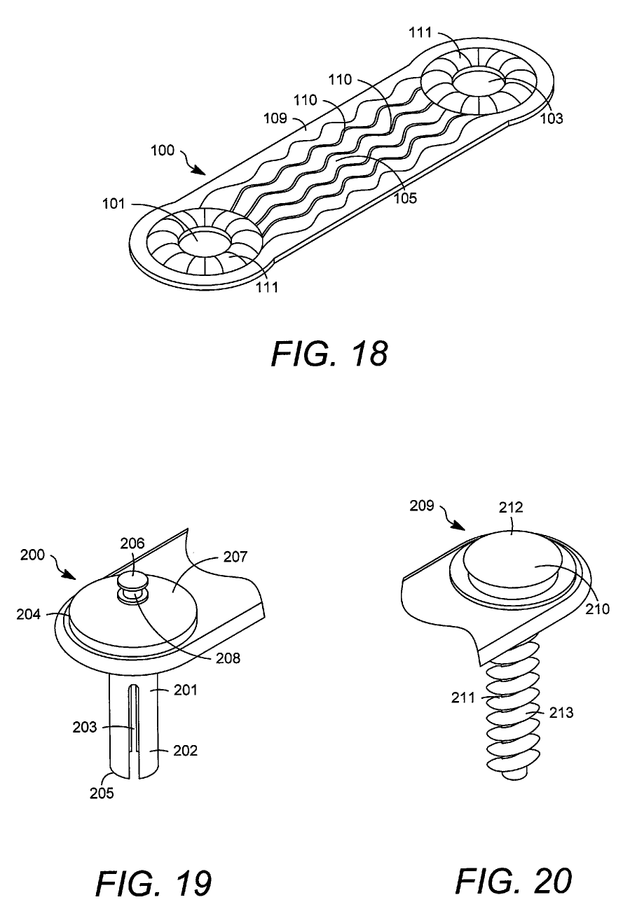

FIG. 18 shows another embodiment of the invention, in which the replacement ligament body 100 is manufactured from a relatively elastic material such as a silicone sheet. The relatively elastic material is formed into a flexible matrix 109, containing reinforcing fibres 110 of a relatively inelastic material such as polyester, or polypropylene that is capable of resisting loads in the EZ range in the same manner as a natural ligament would. Thus, these fibres 110 may be arranged in a woven configuration, orientated in strands or bundles running longitudinally, within the ranges of angles specified elsewhere herein, or in any other appropriate configuration such as in a concertina or wavy fashion, as shown, such that the fibres are effective to accomodate a desired longitudinal elongation under tensile loading expected during load in the NZ range whilst not reacting the applied load. The flexible elastomeric material of the matrix 109 provides for the initial reaction in the NZ load range, during which time the fibers 110 are straightening from their wavy orientation that is shown in the non-tensioned depiction in FIG. 18. As per the previous embodiments, the replacement ligament body 100 consists of a centre section 105 and two ends 101, 104. Each end contains at least one fixation aperture 103. In this embodiment, the replacement ligament design does not need to use grommets, but may contain a hole reinforcement structure 111 inside the flexible matrix 109. The hole reinforcement structure 111 is generally circular (or toroidal), and may be made from the same material as the internal mesh 110, or may be manufactured from an alternative biocompatible material. Its function is to prevent deformation of the fixation apertures 101 and to strengthen the flexible matrix material 109 in the region of the replacement ligament end sections 104, where stress levels are likely to be highest.

Thus, it is appreciated that embodiments such as that depicted in FIG. 18 are within the scope of the invention albeit through components arranged differently than in FIGS. 1 to 17. In embodiments such as that of FIG. 18 a first component deformable responsive to a first, lower load range corresponding to a neutral zone of the natural ligament (such as an ALL) is provided in the replacement ligament body 100, such as by using an elastomeric material as a substantial portion of the matrix 109 of the replacement ligament body 100. Although the fibers 110 may be preconditioned (e.g., preloaded) as described herein, they are arranged in a slack orientation, here in a wavy orientation, the latter not meant to be limiting of various slack orientations known to those skilled in the art. As the load stretchingly deforms the elastomeric material of the matrix 109 the slack is removed. That is, the slack pattern is so adapted, such as by design and calculation, so as to straighten toward a linear pattern during stretching of the flexible matrix 109. Then, at least some of the fibers 110 extend in response to the first part of the more elevated load range corresponding to the elastic zone of the natural ligament. With further load more and more of the fibers 110 may come under load and extend in response to the load. Of course various embodiments may be provided so that essentially all fibers respond to the load across the entire elastic zone. For all such embodiments described in regard to FIG. 18 the respective first and second components (e.g., the elastomeric silicone and the fibers) are aligned along the axis so as to collectively respond to load from movement of the two bones to which they are attached in a manner substantially similarly to the natural ligament being replaced. In these embodiments the replacement ligament body comprises both the first and the second components. It is further appreciated that although the elastomeric material may continue to stretch under load in the elastic zone, the majority of the load in that zone is taken or reacted by the fibers 110 of the second component. Also, based on this and other embodiments' discussion, it is clear that the second component has a different response to load in the first load range (e.g., not under load, or deforming much less than the first component) than in the second load range (where it is under tensile stress from most or all of the load).

In various embodiments of the approaches described above there is no slot or other analogous space in the fixation aperture after insertion of the respective fastener. That is, when a compressible grommet or other compressible insert is used in the various embodiments of this paragraph, there is a close fit between the fastener and the fixation aperture such that there is no slot for movement of the fastener responsive to movement of the bones. However, upon a relative movement of the bones to which a replacement ligament is attached, there is a compression of the compressible grommet or other compressible insert such that a transient space may be formed opposite the compressed region (i.e., on the side opposite the fastener). This is not a slot in the normal meaning of that term.

As noted above, there are embodiments of the invention that use approaches other than, or in addition to, a compressible grommet or other compressible insert for the first component.

In addition to the above embodiments, embodiments of the present invention may include structures in which two or more different types of fibers that are woven together to form the replacement ligament body separately comprise the first and the second components. That is, a first type of fiber may be woven in such a way as to be the first component, and a second and a third fiber together may be woven in such a way as to be the second component.

In addition to the above embodiments, embodiments of the present invention may include structures in which the replacement ligament body is comprised of two or more sections that are assembled together, wherein at least two of such sections have different properties based on their dimensions and/or composition and respectively function separately as the first and the second components.

FIG. 19 and FIG. 20 show two possible fixation devices, although many more are applicable, including staples, darts, and pins. In the first embodiment (FIG. 19), an expanding bollard 200 is used to secure the replacement ligament onto the vertebral body. The bollard 200 is manufactured from a stiff but deformable biocompatible material, such as poly-ether-ether-ketone (PEEK). It consists of a shaft section 201, which passes through the fixation aperture in the replacement ligament, and is driven into a corresponding hole drilled into the vertebral body. The shaft section 201 is cylindrical in shape, and may taper towards the distal end 205, with at least two legs 202 defined by longitudinal slots 203, which extend approximately two-thirds of the way from the distal end of the shaft section 201. The head section 203 of the bollard is dimensioned such that it provides good coverage of the replacement ligament and is of sufficient diameter to ensure that the replacement ligament does not slip over the head section 203 during extension, bending and rotation of the spine. Once the bollard is fully seated (such that the replacement ligament is held securely between the vertebral body and the underside of the head section 203), the central pin 206 is driven into a pre-existing central hole in the bollard 200. The central hole in the bollard 200 is dimensioned so as to be a snug fit for the central pin 206 for the majority of its length, but towards the distal end 205 of the shaft section 201, the central hole diameter decreases, such that the central pin 206, being driven further into the bollard 200, exerts an outward pressure on the shaft section 201 in the region of the bollard legs 202. The central pin 206 is preferably manufactured from a rigid material such as PEEK or titanium alloy or similar, and consequently both it and the shaft section 201 of the bollard 200 will be denser and less compressible than the cancellous bone of the vertebral body surrounding the bollard shaft 201. Consequently, the bollard legs 202 will be inclined to splay radially outwards, crushing regions of the cancellous bone of the vertebral body such that (a) stability of the bollard 200 is achieved, and (b) the effective diameter of the distal end 205 is larger than that of the main shaft section 201 and consequently larger than the initial entry hole drilled through the cortical bone of the vertebral body. This latter condition ensures that the bollard 200 is resistant to backing out due to the loading it will experience after implantation.

If the bollard 200 needs to be removed from the vertebral body at some time post-operatively, the head section 204 contains a recess 207 that provides access to a circumferential groove 208 in the proximal region of the central pin 206. The circumferential groove 208 provides a means for an instrument to grip the central pin 206 and pull it out of the shaft section 201. Once the central pin 206 is removed, the legs 202 can return to their initial position, and the bollard 200 can be removed through its initial entry hole in the vertebral body.

Alternatively, a bone screw may be used, as shown in FIG. 20. The bone screw 209 is made from a proven orthopaedic screw material, such as titanium or stainless steel. It consists of a head section 210 and a shaft section 211. The head section 210 is dimensioned such that it provides good coverage of the replacement ligament and is of sufficient diameter to ensure that the replacement ligament does not slip over the head section 210 during extension, bending and rotation of the spine. The head section 210 also incorporates a drive recess 212, which provides an interface for an instrument to provide a driving torque, in order to tighten the bone screw 209 into the vertebral body. The drive recess 212 may be a hexagonal socket or similar. The shaft section 211 is threaded, preferably with a self-tapping thread 213.

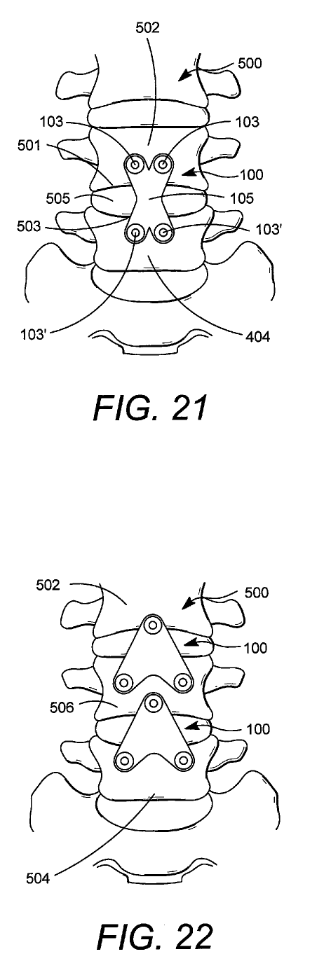

FIG. 21 illustrates typical positioning of the replacement ligament body 100 into the lumbar spine 500. The longitudinal axis of the replacement ligament body 100 is approximately aligned to the longitudinal axis of the lumbar spine 500. The upper fixation apertures 103 of the replacement ligament body 100 are located close to the endplate 501 of the superior vertebral body 502, the lower fixation apertures 103′ of the replacement ligament body 100 are located close to the endplate 503 of the inferior vertebral body 504, and the replacement ligament body's mid-section is centralised over the affected vertebral disc space 505.

FIG. 22 illustrates a potential application for the invention, in addressing the needs of a multiple-level spinal reconstruction. Here, two replacement ligament bodies 100 are attached to a superior vertebral body 502, an inferior vertebral body 504, and an intermediate vertebral body 506. In this application of the invention, the two replacement ligament bodies 100 are separate and can be positioned and tensioned independently.

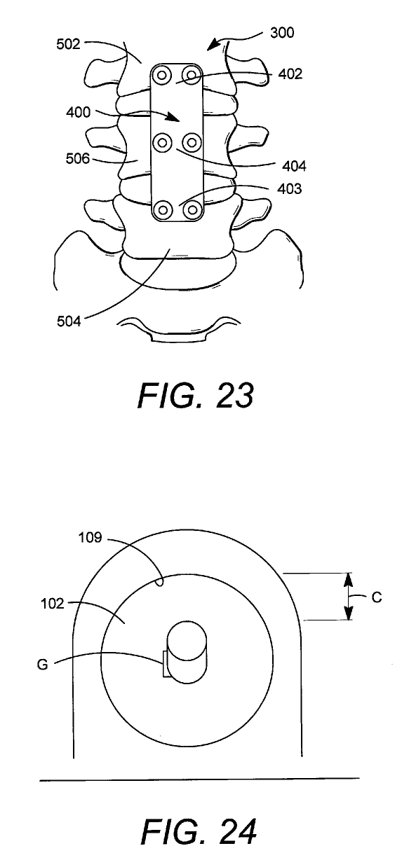

FIG. 23 illustrates a further embodiment of the invention, designed specifically for multiple-level spinal reconstruction. An extended-length replacement ligament 400 includes at least one fixation aperture at each of the superior 402 and inferior 403 end sections, which allow attachment to the superior 502 and inferior 504 vertebral bodies. In addition, the design incorporates at least one intermediate fixation aperture, positioned in the mid-section region 404 of the extended-length replacement ligament 400.

FIG. 24 illustrates the degree by which the compressible grommet 102 will compress with C referencing the compressed side and G referencing the gap which would open up on the opposite side.

According to the stated requirements, the present invention may be provided as a means of restoring stability to a region of the body following reconstructive orthopaedic surgery. As noted initially above in this section, although the initial focus of the invention is as a means of replacing a portion of the anterior longitudinal ligament, it will be appreciated that many of the principles may equally be applied to systems, devices, methods and instruments providing a ligament replacement suited for other bone structures within the human or animal body.

It is further appreciated that the respective arrangements of first and second components, so as to collectively respond to load from movement of two bones to which the ligament system is to attach, each are effective to reduce or eliminate slack in the respective replacement ligament or replacement ligament system, even after prolonged use in the body. Also, in contrast to fusion devices, even those providing for some level of movement such as U.S. Pat. No. 6,206,882, and in contrast to non-supportive barrier devices such as U.S. Pat. No. 6,475,219 which provides an unrestricted relative movement of bones to which it may be attached, the replacement ligaments and replacement ligament systems of the present invention provide ligament-restricted movement. By the latter term, as may be appreciated from the above disclosure, is meant that the movement between bones attached by the present invention devices corresponds to a range of movement that would be permitted by a natural ligament between the bones.

In various embodiments the replacement ligament is non-bioabsorbable, or substantially non-bioabsorbable, so that it will remain functional at a desired performance level for periods of time ranging in tens of years.

It is appreciated that embodiments of the present invention may include additional components so that such embodiments are considered an “engineered tissue.” For example, such “engineered tissue” embodiments may include: isolated cells or cell substitutes infused in a ligament; compounds known to induce tissue growth; and/or cells seeded into tissue scaffolds that are part of the replacement ligament

All patents, patent applications, patent publications, and other publications referenced herein are hereby incorporated by reference in this application in order to more fully describe the state of the art to which the present invention pertains, to provide such teachings as are generally known to those skilled in the art, and to provide such teachings as are noted through references herein.

While various embodiments of the present invention have been shown and described herein, such embodiments are provided by way of example only. Numerous variations, changes and substitutions may be made without departing from the invention herein. Moreover, when any range is described herein, unless clearly stated otherwise, that range is understood to disclose all values therein and all sub-ranges therein, including any sub-range between any two integers within the range, including the endpoints. Accordingly, it is intended that the invention be limited only by the spirit and scope of the appended claims.

1. A replacement ligament comprising:
first and second ends,
two or more securing portions for securing said ligament to an adjacent body portion;
a body portion comprising a first component having a first, higher, elongation per unit load characteristic; and a second component having a second, lower, elongation per unit load characteristic; and wherein
said first and second components are between said two or more securing portions and arranged in load series such that initial, lower, loading is reacted by said first component and subsequent, higher, loading is reacted by said second component.

2. A replacement ligament as claimed in claim 1 wherein said first component comprises a polymer material.

3. A replacement ligament as claimed in claim 2 wherein said first component comprises a plurality of fibres.

4. A replacement ligament as claimed in claim 1 wherein said second component comprises a polymer material.

5. A replacement ligament as claimed in claim 1 wherein said second component comprises a plurality of fibres.

6. A replacement ligament as claimed in claim 1 wherein one or more of said two or more securing portions in said first component comprises a lug having an aperture therein for receiving a bone fixation device.

7. A replacement ligament as claimed in claim 6 wherein said securing portions is a washer and said second filaments are secured thereto.

8. A replacement ligament as claimed in claim 7 wherein said filaments are braided to said lug.

9. A replacement ligament as claimed in claim 6 wherein said first component comprises an elastic material within the aperture of one or more of said lugs.

10. A replacement ligament as claimed in claim 9 wherein said first component comprises a grommet (102) within one or more of said lugs and having an aperture therein for receiving a bone fixator.

11. A replacement ligament as claimed in claim 10 wherein said first and second components comprise filaments and said second filaments have a looser weave than said first filaments such that initial, lower, loading is reacted by said first filaments whilst subsequent, higher, loading is reacted by said second filaments.

12. A replacement ligament as claimed in claim 1 wherein said first component comprises an elastomeric material and said second component comprises a plurality of filaments having a loose weave and said polymer encases said filaments such that initial, lower, loading is reacted by said polymer whilst subsequent, higher, loading is reacted by said filaments.

13. A replacement ligament as claimed in claim 1 wherein said body portion includes a further securing portion between said first and second securing portions, said further securing portion including an aperture formed by a lug.

14. A replacement ligament as claimed in claim 1 wherein said second component comprises woven filaments.

15. A replacement as claimed in claim 14 wherein said filaments comprise a concertina pattern.

16. A replacement ligament as claimed in claim 1 including an anti-adhesive coating on a surface of said filament.

17. A replacement ligament as claimed in 16 including an anti-adhesive layer on top of an outer surface of said filament.

18. A replacement ligament as claimed in claim 17 wherein said coating comprises a tube surrounding said replacement filament.

19. A replacement ligament as claimed in claim 17 wherein at least a portion of said anti-adhesive layer is free floating relative to said ligament.
